SEQUENCE LISTING (27 sequences)

SEQ ID NO: 1 - DNA, 51 bp, Unknown
  Allele
  misc_feature (26)..(26): n = A or G
tagtcccaggaacaaagagagtttgngaatcaatgcctggctaataatagg

SEQ ID NO: 2 - DNA, 51 bp, Unknown
  Allele
  misc_feature (26)..(26): n = C or T
ttgtggaaaaagatagatcaggtccnacttgaagacaaagttgctctcaac

SEQ ID NO: 3 - DNA, 51 bp, Unknown
  Allele
  misc_feature (26)..(26): n = C or T
cagagggagaggacagtaaccaatancgctccttctacatgatcagtgttc

SEQ ID NO: 4 - DNA, 51 bp, Unknown
  Allele
  misc_feature (26)..(26): n = A or G
acctgctggatgctgaattaactgcnatggcaggagagagttacagtcggg

SEQ ID NO: 5 - DNA, 51 bp, Unknown
  Allele
  misc_feature (26)..(26): n = C or T
caagttacttaacctgttggcctatnagttttccctcttgtaaaatggaga

SEQ ID NO: 6 - DNA, 51 bp, Unknown
  Allele
  misc_feature (26)..(26): n = A or G
cctatcctagacctactgaattagantccacattttaataagattctgggt

SEQ ID NO: 7 - DNA, 51 bp, Unknown
  Allele
  misc_feature: n = C or T
  misc_feature (26)..(26): n = C or T
aatggacaaatttgtctgtgatccanatattctctcttcctctagcttagg

SEQ ID NO: 8 - DNA, 51 bp, Unknown
  Allele
  misc_feature (26)..(26): n = A or G
aatatcaaatgggttgggtgagatcncttatgcaatataatcccagcactt

SEQ ID NO: 9 - DNA, 51 bp, Unknown
  Allele
  misc_feature (26)..(26): n = A or G
atgggaaatggaataccataaaattntcatatgttgagcccaaaatgatag

SEQ ID NO: 10 - DNA, 51 bp, Unknown
  Allele
  misc_feature (26)..(26): n = A or G
aatacttgttttctaatgattcaagntacacaaattttatttaatgcacaa

SEQ ID NO: 11 - DNA, 51 bp, Unknown
  Allele
  misc_feature (26)..(26): n = C or G
catacagttggccctctatatccctntatctgtgagttcagtggattcaaa

SEQ ID NO: 12 - DNA, 51 bp, Unknown
  Allele
  misc_feature (26)..(26): n = A or G
ttctgtagttaataaagttaacactntttcatgatggaggctgccccagct

SEQ ID NO: 13 - DNA, 51 bp, Unknown
  Allele
  misc_feature (26)..(26): n = C or T
ttctgggtttgggttagtagtttcangaagatacaccacccttcctcttcc

SEQ ID NO: 14 - DNA, 51 bp, Unknown
  Allele
  misc_feature (26)..(26): n = A or G
acatgctccttcacctttgagcttcngccagggagaaaaacacatattaga

SEQ ID NO: 15 - DNA, 51 bp, Unknown
  Allele
  misc_feature (26)..(26): n = C or T
gagtgtatttgtaaaacatgttgttngccccagtaaatgtattcataaacc

SEQ ID NO: 16 - DNA, 51 bp, Unknown
  Allele
  misc_feature (26)..(26): n = A or G
cacaactaagtgctagggataacatngtgaaaaaattaaaaaacagagaag

SEQ ID NO: 17 - DNA, 51 bp, Unknown
  Allele
  misc_feature (26)..(26): n = C or T
atcgcctacgcctgcagtcagttatncttcactcagaccaccagccctctg

SEQ ID NO: 18 - DNA, 51 bp, Unknown
  Allele
  misc_feature (26)..(26): n = A, C or G
gaaggggttaatggtccccaagcaantctttaacacagcagggcacatatt

SEQ ID NO: 19 - DNA, 51 bp, Unknown
  Allele
  misc_feature (26)..(26): n = A or G
atgaatgcatattgtaaaaccaagcngaggacacagaatggctcctcccac

SEQ ID NO: 20 - DNA, 51 bp, Unknown
  Allele
  misc_feature (26)..(26): n = A or G
tggagaaaccccgtctctaccaaaantataagattagccaggcctggtggg

SEQ ID NO: 21 - DNA, 51 bp, Unknown
  Allele
  misc_feature (26)..(26): n = C or T
caagcatactacagcttaatgtttgnccagatgtgggggaaactttgtttt

SEQ ID NO: 22 - DNA, 51 bp, Unknown
  Allele
  misc_feature (26)..(26): n = C or T
agatcaattgagttaatgtatgtaangtacttggcacagagcttggcccat

SEQ ID NO: 23 - DNA, 51 bp, Unknown
  Allele
  misc_feature (26)..(26): n = A, G or T
taatgtcctaaatctgtaatggagtnaatcaacaagacacttgcaaacaaa

SEQ ID NO: 24 - DNA, 51 bp, Unknown
  Allele
  misc_feature (26)..(26): n = A or G
actcagtttctttacatagctatagnatgagggcgtagtcctagagtgctc

SEQ ID NO: 25 - DNA, 51 bp, Unknown
  Allele
  misc_feature (26)..(26): n = G or T
tgttgtgaagattaaatgagatggtncttaaatgttacttagtagtagtag

SEQ ID NO: 26 - DNA, 51 bp, Unknown
  Allele
  misc_feature (26)..(26): n = A or G
catgggtctttagaagcattaggaanaggcaacacagcgtggggtcctcag

SEQ ID NO: 27 - DNA, 51 bp, Unknown
  Allele
  misc_feature (26)..(26): n = A or G
ctgcaaggggcatccgtggcgaaccnaaagatctctcagttgaagaccagg

(19) United States
(12) Patent Application Publication
(10) Pub. No.: US 2022/0145354 A1
(43) Pub. Date: May 12, 2022

(54) METHOD AND SYSTEM FOR PREDICTING RESPONSE OF A SUBJECT TO ANTIDEPRESSANT TREATMENT

(71) Applicant: TALIAZ LTD., Tel Aviv (IL)

(72) Inventor: Dekel TALIAZ, Modi'in (IL)

(21) Appl. No.: 17/585,751

(22) Filed: Jan. 27, 2022

Related U.S. Application Data
(63) Continuation of application No. 16/343,852, filed on Apr. 22, 2019.
(60) Provisional application No. 62/414,749, filed on Oct. 30, 2016.
(60) Provisional application No. 62/414,750, filed on Oct. 30, 2016.

(51) Int. Cl. C12Q 1/68; C12Q 1/6883; G16B 40/00; G16H 10/20; A61P 25/24; G16H 50/30; G16H 20/10; G16B 40/20; G16H 10/60; G16H 50/20; G16B 30/00

(52) U.S. Cl. CPC C12Q 1/68; C12Q 1/6883; G16B 40/00; G16H 10/20; A61P 25/24; G16H 50/30; C12Q 2600/106; G16B 40/20; G16H 10/60; G16H 50/20; G16B 30/00; C12Q 2600/156; G16H 20/10

(57) ABSTRACT

Methods for predicting antidepressant treatment response for a subject in need thereof, for predicting resistance to antidepressant treatment, and for generating a predictor of response to antidepressant treatment, are provided.

### RELATED APPLICATIONS

This application is a continuation of U.S. Ser. No. 16/343,852 filed on Apr. 22, 2019, which is a National Phase of PCT Patent Application No. PCT/IL2017/051177 having International filing date of Oct. 30, 2017, which claims the benefit of priority of U.S. Provisional Application Nos. 62/414,749 and 62/414,750, both filed on Oct. 30, 2016. The contents of the above applications are all incorporated by reference as if fully set forth herein in their entirety.

### FIELD OF THE INVENTION

The invention is directed to systems and methods for predicting response and resistance to antidepressant treatment in a subject in need thereof.

### BACKGROUND OF THE INVENTION

Mood disorders are among the most prevalent forms of mental illness. Severe forms of mental illness affect 2%-5% of the US population and up to 20% of the population suffers from milder forms of the illness (Nestler et al., 2002, Neuron 34, 13-25). The economic costs to society and personal costs to individuals and families are enormous.

Anti-depressants are a primary method for treatment of depression. Anti-depressant drugs are known to influence the functioning of certain monoamine neurotransmitters, primarily serotonin, norepinephrine, and dopamine. Older medications, such as tricyclic anti-depressants (TCAs) and monoamine oxidase inhibitors (MAOIs), affect the activity of all these neurotransmitters simultaneously. However, these medications can be difficult to tolerate due to side effects or, in the case of MAOIs, dietary and medication restrictions. Newer medications, such as selective serotonin reuptake inhibitors (SSRIs) norepinephrine reuptake inhibitors (NRIs), Serotonin-norepinephrine reuptake inhibitors (SNRIs), Norepinephrine-Dopamine Reuptake Inhibitors (NDRIs) and Serotonin-Norepinephrine-Dopamine Reuptake Inhibitors (SNDRIs), Mirtazapine, Nefazodone, Trazodone, and Vortioxetine also have side-effects, though fewer. Prescription of anti-depressant medication is often inexact and their efficacy is assessed empirically. Depression, as well as other prevalent psychiatric disorders, is characterized by a high degree of variability in patient response to the drugs administered, even among individuals with the same diagnosis. In fact, only roughly 35% of patients demonstrate complete remission following first prescribed treatment. Furthermore, some patients respond, but with serious adverse side effects (Nestler et al., 2002, Neuron 34, 13-25).

Current methods for selecting a suitable depression treatment are basically trial-and-error. Patients will often have to be treated with several kinds of medicine, before finding the most suitable drug. This is obviously a problem, which is further augmented by the fact that four to six weeks of chronic treatment are required to evaluate the anti-depressant phenotype, the efficacy of the treatment and whether an adverse event is registered. It is therefore not surprising that patients tend to cease taking their medications against medical advice.

Forty to fifty percent of the risk for depression is genetic making depression a highly heritable disorder. However, the search for a single gene responsible for major depressive disorder has given way to the understanding that depression is a complex disease in which multiple gene variants, each having only a slight contribution to the disorder, are involved (Nestler et al., 2002, Neuron 34, 13-25). The explanation to the variation in treatment efficacy of the different anti-depressants is also most probably due to the different genetic background of the patients. However, the search for gene variants explaining this variance has been of limited success.

U.S. Pat. No. 6,399,310 discloses methods for improving the therapeutic response of human patients with major depression by determining the apolipoprotein E genotype of a human patient and administering mirtazapine, in an amount effective to treat major depression, to those patients who are found to carry a certain genotype of the gene for apolipoprotein E4.

US 2006/0160119 discloses a method for screening patients to determine whether or not SSRI therapy is likely to alleviate symptoms of depression in those patients. The method provides a polymorphism at position −1019 of the 5-HT1A gene that is predictive of likelihood of improvement of symptoms and a polymorphism at position 102 of the 5-HT2A gene that is predictive of likelihood of unwanted side effects related to SSRI therapy administered to a patient.

US 2005/0069936 discloses methods for the diagnosis and evaluation of depression treatment. In particular, patient test samples are analyzed for the presence and amount of members of a panel of markers comprising one or more specific markers for depression treatment and one or more non-specific markers for depression treatment. A variety of markers are disclosed for assembling a panel of markers for such diagnosis and evaluation. Algorithms for determining proper treatment are disclosed. In various aspects, the invention provides methods for the early detection and differentiation of depression treatment.

However, there remains an unmet medical need for a method capable of predicting the efficacy and adverse effects of an anti-depressant treatment in a patient suffering from a psychiatric disorder such as depression, taking into account clinical as well as genetic information. This is important in order to shorten the time required to achieve an optimal treatment regime with minimal adverse side effects.

### SUMMARY OF THE INVENTION

The invention is directed to methods, devices and systems for predicting efficacy and adverse effects of an anti-depressant treatment in a patient suffering from a psychiatric disorder, such as depression. The invention is also directed to methods, devices and systems for predicting treatment resistance to anti-depressants of a subject suffering from a psychiatric disorder.

Advantageously, the methods, devices and system disclosed herein enable predicting the patient's response to treatment with antidepressants, as well as predicting the patient being treatment-resistant prior to the patient being treated with antidepressants. That is, the methods enable predicting the patient's treatment response and/or the patient as being treatment resistant based on a combination of clinical and genetic data, wherein the clinical data may be obtained, for example, through a patient' answers to a questionnaire. In particular embodiments, the methods of the invention are exemplified herein for prediction of treatment resistance, and for the prediction of efficacy and side effects to citalopram (CELEXA™, CIPRAMIL®) and venlafaxine (EFFEXOR™).

The present invention provides, in one aspect, a method for predicting citalopram treatment response for a subject in need thereof, the method comprising obtaining at least one clinical feature of the subject, obtaining a sample comprising genetic material from the subject, detecting a nucleotide identity of at least two polymorphic sites in the genetic material, and processing the at least one clinical feature and the nucleotide identity of the at least two polymorphic sites by applying a classification algorithm, the classification algorithm configured to provide a graduated score indicative of the treatment response to citalopram.

According to some embodiments, the at least one clinical feature is selected from the group consisting of: severity level of problems in the upper gastro intestine, pains or aches at different body parts, reported fear of having anxiety attack, history of psychotropic medications, poor treatment response to other antidepressants, reported troubling thoughts, employment status, residence, private health care insurance and any combination thereof.

In certain embodiments, the at least one clinical feature is selected from the group consisting of severity level of problems in the upper gastro intestine, reported pains or aches in different body parts, reported fear of having an anxiety attack, having a history of psychotropic medications, having a poor treatment response to other antidepressants, reported troubling thoughts, and any combination thereof.

In certain embodiments, the at least one clinical feature is a plurality of clinical features, comprising severity level of problems in the upper gastro intestine, reported pains or aches in different body parts, reported fear of having an anxiety attack, having a history of psychotropic medications, having a poor treatment response to other antidepressants and reported troubling thoughts.

In certain embodiments, the method further comprises obtaining personal and/or demographic information regarding the subject.

In certain embodiments, the personal information obtained relates to employment status, having private healthcare insurance, age, marital status, residence, and any combination thereof.

In certain embodiments, the personal information obtained relates to employment status and having private healthcare insurance.

According to some embodiments, at least two polymorphic sites are selected from the group consisting of: rs17291388, rs558025, rs7201082 and any combination thereof.

According to some embodiments, the method further includes detecting a nucleotide identity of at least three polymorphic sites, the at least three polymorphic sites comprising rs17291388, rs558025 and rs7201082.

In certain embodiments, the at least two polymorphic sites are a plurality of polymorphic sites, comprising rs17291388, rs558025, and rs7201082.

According to some embodiments, the method includes obtaining at least three clinical features.

According to some embodiments, the method further comprises determining sexual side effects of the citalopram treatment. According to some embodiments, determining sexual side effects of the citalopram treatment comprises obtaining the patients' age and gender and applying the classification algorithm thereon.

According to some embodiments, the classification algorithm comprises a non-linear classification algorithm. According to some embodiments, the non-linear classification algorithm comprises an ensemble of classification and regression trees. According to some embodiments, the ensemble of classification and regression trees, comprises a random forest classifier or a boosting framework.

According to some embodiments, the graduated score has an accuracy of above 0.5 and a p-value for the accuracy of below 0.05 and an AUC of above 0.5. According to some embodiments, the graduated score has an accuracy of above 0.6 and a p-value for the accuracy of below 0.01 and an AUC of above 0.6.

According to some embodiments, the graduated score has an accuracy of at least 0.62295 with a p-value of 2.95E-06 and an AUC of at least 0.6589.

According to some embodiments, the graduated score has an accuracy of at least 0.775 with a p-value of 0.001 and an AUC of at least 0.8511

The present invention further provides, in an aspect, a method for identifying a subject as being resistant to antidepressant treatment, the method comprising obtaining at least one clinical feature of the patient, obtaining a sample comprising genetic material from the subject, detecting a nucleotide identity of at least two polymorphic sites in the genetic material, and processing the at least one clinical feature and the nucleotide identity of the at least two polymorphic sites by applying a classification algorithm, the classification algorithm configured to provide a graduated score indicative of the subject's treatment resistance.

According to some embodiments, the at least one clinical feature is selected from the group consisting of: severity level of problems in the musculoskeletal/integument system, severity level of problems in the neurological system, employment status, resistance, age, feared of having an anxiety attack, reported feeling of unease and any combination thereof.

According to some embodiments, the at least one clinical feature is selected from the group consisting of: severity level of problems in the musculoskeletal/integument system, severity level of problems in the neurological system, avoiding doing something because of fear from having an anxiety attack, fear of having an anxiety attack when traveling in a bus, train, or plane, being jumpy and easily startled because of having experienced a traumatic event, and any combination thereof.

According to some embodiments, the at least one clinical feature is a plurality of clinical features, comprising severity level of problems in the musculoskeletal/integument system, severity level of problems in the neurological system, avoiding doing something because of fear from having an anxiety attack, fear of having an anxiety attack when traveling in a bus, train, or plane, employment status, resistance, age, and being jumpy and easily startled because of having experienced a traumatic event.

According to some embodiments, the method further comprises obtaining personal and/or demographic information regarding the subject.

According to some embodiments, the personal information obtained relates to employment status, residence, age, having private healthcare insurance, marital status, and any combination thereof.

According to some embodiments, the personal information obtained relates to employment status, residence and age.

According to some embodiments, the at least two polymorphic sites are selected from the group consisting of: rs1057079, rs10892629, rs12625531, rs1303860, rs1349620, rs1361038, rs1475774, rs1488467, rs16912741, rs16959216, rs17049528, rs1854696, rs1873906, rs1891932, rs3122155, rs4845882, rs530296, rs625109, rs6913639, rs7203315, rs732123, rs948025 and any combination thereof.

According to some embodiments, the at least two polymorphic sites are a plurality of polymorphic sites, comprising rs1057079, rs10892629, rs12625531, rs1303860, rs1349620, rs1361038, rs1475774, rs1488467, rs16912741, rs16959216, rs17049528, rs1854696, rs1873906, rs1891932, rs3122155, rs4845882, rs530296, rs625109, rs6913639, rs7203315, rs732123, and rs948025.

According to some embodiments, the method comprises detecting a nucleotide identity of at least four polymorphic sites. According to some embodiments, the method comprises detecting a nucleotide identity of at least ten polymorphic sites.

According to some embodiments, resistance to antidepressant treatment comprises resistance to at least two of the antidepressant medications selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone and vortioxetine and any combination thereof.

According to some embodiments, the method comprises obtaining at least three clinical features.

According to some embodiments, the classification algorithm comprises a non-linear classification algorithm. According to some embodiments, the non-linear classification algorithm comprises an ensemble of classification and regression trees. According to some embodiments, the ensemble of classification and regression trees, comprises a random forest classifier or a boosting framework.

According to some embodiments, the graduated score has an accuracy of above 0.5 and a p-value for the accuracy of below 0.05 and an AUC of above 0.5.

According to some embodiments, the graduated score has an accuracy of at least 0.5998 with a p-value of 7.24E-06 and an AUC of at least 0.6381

The present invention further provides methods and kits for predicting treatment efficacy of venlafaxine in a subject in need thereof.

The methods of the invention are based in part on the unexpected discovery of two SNP, namely, rs2283351 in CCDC63 gene and rs10497340 in the gene locus LOC102724081, which are highly associated with a subject's response to treatment with venlafaxine. The present invention shows the ability of rs2283351 and rs10497340, separately or in combination, to reliably predict venlafaxine treatment response. Accordingly, the present invention provides for the first time a strong predictive platform to help physicians in deciding whether to prescribe venlafaxine to a subject, or not.

The aforementioned surprising discoveries are a result of using advanced machine learning techniques in combination with expert knowledge in methods described herein.

According to some embodiments, there is provided a method for predicting a subject's response to treatment with venlafaxine, comprising: obtaining a sample comprising genetic material from the subject; and detecting a nucleotide identity of rs2283351 or rs10497340 in the genetic material.

According to some embodiments, there is provided a method for predicting responsiveness to venlafaxine treatment of a subject in need of antidepressant treatment, the method comprising obtaining a sample comprising genetic material from the subject; and identifying the identity of a nucleotide of polymorphic site rs2283351 in the genetic material.

According to some embodiments, the method further comprises identifying a nucleotide-identity of the polymorphic site rs10497340 in the genetic material.

According to some embodiments, the method further comprises processing the identified nucleotide identity of rs2283351 and rs10497340 by applying a classification algorithm, the classification algorithm is configured to provide a graduated score indicative of the treatment response to venlafaxine.

According to some embodiments, the method further comprises prescribing and/or administering venlafaxine to the subject if the nucleotide identity of polymorphic site rs2283351 is adenine/adenine. According to some embodiments, the method further comprises prescribing and/or administering venlafaxine to the subject if the nucleotide identity of polymorphic site rs2283351 is guanine/guanine. According to some embodiments, the method further comprises prescribing and/or administering venlafaxine to the subject if the nucleotide identity of polymorphic site rs2283351 is guanine/guanine and the nucleotide identity of polymorphic site rs10497340 is guanine/guanine. According to some embodiments, the method further comprises prescribing and/or administering venlafaxine to the subject if the nucleotide identity of polymorphic site rs2283351 is adenine/adenine and the nucleotide identity of polymorphic site rs10497340 is guanine/guanine. According to some embodiments, the method further comprises prescribing and/or administering venlafaxine to the subject if the nucleotide identity of polymorphic site rs2283351 is adenine/adenine and the nucleotide identity of polymorphic site rs10497340 is adenine/guanine.

According to some embodiments, the method further comprises refraining from prescribing and/or administering venlafaxine to the subject if the nucleotide identity of polymorphic site rs2283351 is guanine/adenine. According to some embodiments, the method further comprises refraining from prescribing and/or administering venlafaxine to the subject if the nucleotide identity of polymorphic site rs2283351 is guanine/guanine and the nucleotide identity of polymorphic site rs10497340 is adenine/guanine. According to some embodiments, the method further comprises refraining from prescribing and/or administering venlafaxine to the subject if the nucleotide identity of polymorphic site rs2283351 is adenine/guanine and the nucleotide identity of polymorphic site rs10497340 is guanine/guanine. According to some embodiments, the method further comprises refraining from prescribing and/or administering venlafaxine to the subject if the nucleotide identity of polymorphic site rs2283351 is adenine/guanine and the nucleotide identity of polymorphic site rs10497340 is adenine/guanine.

According to some embodiments, the method further comprises amplifying genetic loci encompassing said polymorphic sites.

According to some embodiments, the sample is obtained from a biological specimen selected from the group consisting of: blood, saliva, urine, sweat, buccal material, skin and hair. Each possibility represents a separate embodiment of the present invention.

According to some embodiments, the subject in need of antidepressant treatment is diagnosed with depression.

According to some embodiments, the prediction of the responsiveness to venlafaxine treatment is performed prior to initiation of venlafaxine treatment.

According to some embodiments, predicting the subject's responsiveness to venlafaxine treatment comprises predicting the subject's responsiveness to venlafaxine treatment with an accuracy of at least 75%.

According to some embodiments, there is provided a kit for predicting responsiveness to venlafaxine treatment of a subject in need of antidepressant treatment, comprising oligonucleotides for amplification of a genetic loci encompassing polymorphic site rs2283351 in a sample of genetic material obtained from the subject.

According to some embodiments, the kit further comprises oligonucleotides for amplification of a genetic loci encompassing polymorphic site rs10497340 in a sample of genetic material obtained from the subject.

According to some embodiments, the kit further comprises computer readable software configured to enable processing of the identified nucleotide identity of rs2283351 and/or rs10497340 by applying a classification algorithm, the classification algorithm configured to provide a graduated score indicative of the treatment response to venlafaxine.

According to some embodiments, the kit further comprises means for determining the presence of a guanine/adenine in the polymorphic site of rs2283351 and/or guanine/adenine in the polymorphic site of rs10497340.

According to some embodiments, the kit further comprises means for extracting the genetic material from a biological specimen obtained from the subject.

According to some embodiments, the biological specimen is selected from the group consisting of: blood, saliva, urine, sweat, buccal material, skin and hair.

According to some embodiments, the kit further comprises means for acquiring the biological specimen from the subject.

According to some embodiments, the subject is diagnosed with depression.

The present invention further provides, in another aspect, a method for generating a predictor of antidepressant treatment, the method comprising selecting genomic and clinical features relevant to a subject's response to the antidepressant treatment based on expert knowledge, biological models and feature selection algorithms; ranking the selected features based on feature meta-ranking and/or one or more machine learning algorithms; and generating an ensemble predictor based on the feature selection and feature ranking.

The present invention further provides, in another aspect, a method for generating a predictor of antidepressant treatment, the method comprising selecting genomic and clinical features relevant to a subject's response to the antidepressant treatment based on expert knowledge, biological models and feature selection algorithms; ranking the selected features based on feature meta-ranking and/or one or more machine learning algorithms; generating an ensemble predictor based on the feature selection and/or feature ranking; and evaluating the ensemble predictor based on exponential modeling of the subject's treatment response, the exponential modeling based on an integrated analysis of changes in the subject's depression score and duration of treatment.

According to some embodiments, the initial ranking of selected genomic and clinical features is based on meta-analysis and is further revised based on the outcome of treatment versus predicted response.

According to some embodiments, the method further includes evaluating the ensemble predictor, based on exponential modeling of the subject's treatment response. According to some embodiments, the exponential modeling is based on an integrated analysis of changes in the subject's depression score and the duration of treatment.

According to some embodiments, the evaluation of ensemble predictor may (additionally or alternatively) be based on the subject's treatment response, wherein an improvement of at least 50% in the subject's depression score, after as compared to before treatment, is indicative of the subject being responsive to the treatment.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a flow-chart of a prediction method, according to some embodiments;

FIG. 2A shows modeling of a patient's response to antidepressant treatment based on the depression score measured before and after treatment;

FIG. 2B shows exponential modeling of a patient's response to antidepressant treatment.

FIG. 3 depicts the efficacy (sensitivity and specificity) of the ensemble predictor in predicting venlafaxine treatment response.

FIG. 4 depicts the performance of the ensemble predictor as a predictor of venlafaxine treatment efficacy.

### DETAILED DESCRIPTION OF THE INVENTION

In the following description, various aspects of the disclosure will be described. For the purpose of explanation, specific configurations and details are set forth in order to provide a thorough understanding of the disclosure. However, it will also be apparent to one skilled in the art that the embodiments may be practiced without specific details being presented herein. Furthermore, well-known features may be omitted or simplified in order not to obscure the invention.

As used herein, the term “anti-depressant treatment” refers to drugs used in the treatment of patients suffering from depression. Antidepressants are known to influence the functioning of certain monoamine neurotransmitters, primarily serotonin, norepinephrine, and dopamine. Older medications, such as tricyclic anti-depressants (TCAs) and monoamine oxidase inhibitors (MAOIs), affect the activity of all these neurotransmitters simultaneously. Newer medications comprise selective serotonin reuptake inhibitors (SSRIs), norepinephrine-selective reuptake inhibitors (NRIs), Norepinephrine-Dopamine Reuptake Inhibitors (NDRIs), Serotonin-Norepinephrine-Dopamine Reuptake Inhibitor (SNDRI), Mirtazapine, Nefazodone, Trazodone, and Vortioxetine.

As used herein, the term “psychiatric disorders” refers to any psychiatric disorders including, but not limited to, depression, attention deficit disorder, schizophrenia, bipolar disorder, anxiety disorders, alcoholism, eating disorders such as anorexia and bulimia, phobias, dissociative disorders, insomnia, and borderline personality disorder.

As used herein, the terms “depression,” “depressive disorder,” and “mood disorder” interchangeably refer to a DSM-IV definition of depression. It is to be understood that depression comprises different subtypes such as Atypical depression (AD), Melancholic depression, Psychotic major depression (PMD), Catatonic depression, Postpartum depression (PPD), Seasonal affective disorder (SAD), Dysthymia, Depressive Disorder Not Otherwise Specified (DD-NOS), Recurrent brief depression (RBD), Major depressive disorder and Minor depressive disorder; which all fall under the scope of the invention.

Atypical depression (AD) is characterized by mood reactivity (paradoxical anhedonia) and positivity, significant weight gain or increased appetite (“comfort eating”), excessive sleep or somnolence (hypersomnia), a sensation of heaviness in limbs known as leaden paralysis, and significant social impairment as a consequence of hypersensitivity to perceived interpersonal rejection.

Melancholic depression is characterized by a loss of pleasure (anhedonia) in most or all activities, a failure of reactivity to pleasurable stimuli, a quality of depressed mood more pronounced than that of grief or loss, a worsening of symptoms in the morning hours, early-morning waking, psychomotor retardation, excessive weight loss, or excessive guilt.

Psychotic major depression (PMD), or simply psychotic depression, is the term for a major depressive episode, in particular of melancholic nature, wherein the patient experiences psychotic symptoms such as delusions or, less commonly, hallucinations.

Catatonic depression is a rare and severe form of major depression involving disturbances of motor behavior and other symptoms. Here, the person is mute and almost stuporose, and either is immobile or exhibits purposeless or even bizarre movements.

Postpartum depression (PPD) refers to the intense, sustained and sometimes disabling depression experienced by women after giving birth.

Seasonal affective disorder (SAD), also known as “winter depression” or “winter blues”, refers to depressive episodes coming on in the autumn or winter, and resolving in spring.

Dysthymia is a chronic, different mood disturbance where a person reports a low mood almost daily over a span of at least two years. The symptoms are not as severe as those for major depression.

Depressive Disorder Not Otherwise Specified (DD-NOS) refers to disorders that are impairing but do not fit any of the officially specified diagnoses.

Recurrent brief depression (RBD) is distinguished from major depressive disorder primarily by differences in duration. People with RBD have depressive episodes about once per month, with individual episodes lasting less than two weeks and typically less than 2-3 days.

As used herein, the term “gene” has its meaning as understood in the art. In general, a gene is taken to include gene regulatory sequences (e.g. promoters, enhancers, etc.) and/or intron sequences, in addition to coding sequences (open reading frames). It will further be appreciated that definitions of “gene” include references to nucleic acids that do not encode proteins but rather encode functional RNA molecules such as microRNAs (miRNAs), tRNAs, etc. The term “genetic material” as used herein includes RNA or DNA each in double or single stranded form, and functional equivalents thereof.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotide” which comprise at least one modification, including, for example, analogous linking groups, purine, pyrimidines, and sugars. However, the polynucleotides of the invention are preferably comprised of greater than 50% conventional deoxyribose nucleotides, and most preferably greater than 90% conventional deoxyribose nucleotides The polynucleotide sequences of the invention may be prepared by any known method, including synthetic, recombinant, ex vivo generation, or a combination thereof, as well as utilizing any purification methods known in the art.

As used herein, the term “allele” refers to an alternative version (i.e., nucleotide sequence) of a gene or DNA sequence at a specific chromosomal locus.

The term “primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified. Primers used to carry out this invention are designed to be substantially complementary to each strand of the genomic locus to be amplified. This means that the primers must be sufficiently complementary to hybridize with their respective strands under conditions which allow for the polymerization to occur. In other words, the primers should have sufficient complementarity with the 5′ and 3′ sequences flanking the mutation to hybridize therewith and permit amplification of the genomic locus. It should be noted that certain techniques, such as the Sanger sequencing method, make use of only one primer.

As used herein, the terms “sequence variant”, “polymorphism” and “mutation” are used interchangeably and encompass any sequence differences between two nucleic acid molecules. There are many such sites within each genome. For example, the human genome exhibits sequence variations which occur on average every 500 base pairs. Polymorphic markers include restriction fragment length polymorphisms, variable number of tandem repeats (VNTRs), hypervariable regions, minisatellites, dinucleotide repeats, trinucleotide repeats, tetranucleotide repeats, simple sequence repeats, and insertion elements such as Alu. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles at each SNP site; the original allele and the mutated allele. Due to natural genetic drift and possibly also selective pressure, the original mutation has resulted in a polymorphism characterized by a particular frequency of its alleles in any given population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general, polymorphisms can comprise any number of specific alleles within the population, although each human individual has two alleles at each polymorphic site. All such polymorphisms can be utilized in the methods and kits of the present invention, and are thus within the scope of the invention. Sequence variants can be found in a coding sequence of a gene, in a non-coding sequence of a gene, in a regulatory sequence of a gene, in a promoter sequence of a gene as well as in DNA sequences unrelated to genes and/or regulatory elements, such as junk DNA and any combination thereof. As used herein, the term “polymorphic site” refers to the position, in a nucleotide sequence, of the nucleotide that varies among individuals.

As used herein, the term “correlating” as used herein refers to comparing the presence of specific gene variations and/or specific clinical observations in a patient to their presence in individuals known to respond to a certain treatment; or in individuals known to be free of a given condition, i.e. “normal individuals”.

As used herein the term “clinical feature” may refer to any non-genetic parameter influencing the subject's response to an antidepressant treatment. According to some embodiments, the term “clinical feature” may include physiological features (e.g. pain), psychological features (anxiety) as well as sociological features (e.g. marital status) and economical features (e.g. salary), as further described herein below.

It is to be understood that “responsive” to venlafaxine as used herein does not necessarily mean that the subject will benefit from the treatment with venlafaxine, but rather that the subject is, in a statistical sense, more likely to belong to the class of patients that will benefit from the venlafaxine treatment.

The term “classification algorithm” as used herein refers to methods that implement a model (classifier) for predicting a discrete category or class membership (target label), to which the data belong.

The term “non-linear classification algorithm” as used herein refers to non-linear models (classifiers) for prediction of class membership (target label).

The term “classification tree” as used herein refers to a non-linear model (classifier) for predicting class membership (target label) by constructing a decision tree, which repeatedly partitions the data, until it reaches a prediction of a discrete class (target labels).

The term “regression tree” as used herein refers to a non-linear method for predicting numerical values (target values). It involves constructing a decision tree for repeatedly partitioning the data, and predicting real-number target values.

The term “graduated score” as used herein refers to the total score that each subject receives from the method, which quantifies the predicted outcome. An accuracy above 0.5, represents a higher than the random chance of obtaining correct predictions. A p-value for the accuracy below 0.05, refers to a threshold used to limit the likelihood for false negative predictions to no more than 5% of the total number of predictions. The area under the curve (AUC) is used to determine which of the used models best classifies the data (target labels). An AUC of 0.5 is equal to a random prediction, whereas an AUC above 0.5, represent predictions where the true positive rates are greater than those that would be obtained by chance, and the false positive rates are minimized.

The term “random forest classifier” as used herein refers to a non-linear method for predicting a class membership (target label) by constructing multiple decision trees, and predicting the target labels based on the majority vote of the decision trees.

The term “boosting framework” as used herein refers to a method that combines multiple weak prediction models, which are sequentially added and weighted to produce a strong prediction model. This overall stronger model is used to predict the target values, in the case of regression, or target labels in the case of classification.

The term “sexual side effects” as used herein refers to side effects that can be caused by medication, which cause sexual dysfunction, decreased sexual desire, decreased sexual response and/or sexual ability.

As used herein, the term “efficacy” with regards to a subject's response to an antidepressant treatment refers to an improvement of 50% or more in the subject's depression score. Additionally or alternatively, the efficacy may be determined according to a depression curve taking into consideration both the depression score as well as time of treatment. The efficacy of the anti-depressant treatment is determined quantitatively by one or more rating scales, such as the Hamilton Rating Scale for Depression (HAM-D), QUICK INVENTORY OF DEPRESSIVE SYMPTOMATOLOGY (QIDS), Beck's Depression Inventory (BDI), Emotional State Questionnaire or Global Clinical Impression Scale. The HAM-D scale contains items that assess somatic symptoms, insomnia, working capacity and interest, mood, guilt, psychomotor retardation, agitation, anxiety, and insight. As used herein a 50% decrease in the HAM-D or the BDI score is considered an efficient treatment response. The degree of adverse side effects of anti-depressant treatment is determined quantitatively by the Udvalg Kliniske Undersøgelser (UKU) Side Effect Rating Scale, the Frequency and Intensity of Side Effects Rating (FISER) or the Global Rating of Side Effects Burden (GRSEB) scales. Each possibility is a separate embodiment of the invention.

As used herein, the term “treatment resistant” or “resistant to antidepressant treatment” refers to a subject being unresponsive to at least two different antidepressant medications, such as but not limited to SSRIs such as but not limited to: citalopram, paroxetine, sertraline, zimelidine, escitalopram, indalpine, dapoxetine, fluvoxamine and fluoxetine; NRIs such as but not limited to: talopram, talsupram, reboxetine, viloxazine and atomoxetine; NDIRs such as but not limited to bupropion and desoxypipradrol; SNRIs such as but not limited to: edivoxetine, amedalin, desvenlafaxine, milnacipram, daledalin, venlafaxine, duloxetine, tandamine, lortalamine and levomilnacipran; piperidines such as but not limited to difemetorex and dexmethylphenidate; tetracyclic antidepressants such as but not limited to maprotiline and atypical antidepressants, such as, but not limited to Mirtazapine, Nefazodone, Trazodone, and Vortioxetine.

According to some embodiments, there is provided a method for predicting treatment efficacy of a psychiatric drug in a subject in need thereof, the method including obtaining a sample comprising genetic material from the subject; detecting a nucleotide identity of at least one polymorphic site in the genetic material; and processing the nucleotide identity of the at least one polymorphic sites by applying a classification algorithm, the classification algorithm configured to provide a graduated score indicative of the treatment response to the psychiatric drug.

According to some embodiments, there is provided a method for predicting treatment efficacy of a psychiatric drug in a subject in need thereof, the method including at least one clinical feature of the subject; obtaining a sample comprising genetic material from the subject; detecting a nucleotide identity of at least one polymorphic site in the genetic material; and processing the at least one clinical feature and the nucleotide identity of the at least one polymorphic sites by applying a classification algorithm, the classification algorithm configured to provide a graduated score indicative of the treatment response to the psychiatric drug.

According to some embodiments, there is provided a method for predicting treatment efficacy of a psychiatric drug in a subject in need thereof, the method including obtaining at least one clinical feature of the subject; obtaining a sample comprising genetic material from the subject; detecting a nucleotide identity of at least two polymorphic sites in the genetic material; and processing the at least one clinical feature and the nucleotide identity of the at least two polymorphic sites by applying a classification algorithm, the classification algorithm configured to provide a graduated score indicative of the treatment response to the psychiatric drug.

According to some embodiments, the subject in need of the psychiatric drug may suffer from a psychiatric disorder selected from the group consisting of depression, attention deficit disorder, schizophrenia, bipolar disorder, anxiety disorders, alcoholism, eating disorders such as anorexia and bulimia, phobias, dissociative disorders, insomnia, and borderline personality disorder or any combination thereof. According to some embodiments, the subject is suffering from depression and the psychiatric drug is an anti-depressant. According to some embodiments, the antidepressant is selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone, and vortioxetine and any combination thereof. According to some embodiments, the anti-depressant is citalopram.

According to some embodiments, the at least one clinical feature is selected from the group consisting of: severity level of problems in the upper gastro intestine, pains or aches at different body parts, reported fear of having anxiety attack, history of psychotropic medications, poor treatment response to other antidepressants, reported troubling thoughts, fear of illness, employment status, residence, private health care insurance and any combination thereof. According to some embodiments, the method includes obtaining at least three of the above-mentioned clinical features. According to some embodiments, the clinical features incorporated into the method may include most or all of the above-described clinical features.

According to some embodiments, the at least one and/or the at least two polymorphic sites are selected from the group consisting of: rs17291388, rs558025, rs7201082 and any combination thereof. According to some embodiments, the at least one and/or the at least two polymorphic sites include rs17291388, rs558025 and rs7201082.

rs17291388 is set for in SEQ ID NO. 1 (polymorphic
site bracketed):
TAGTCCCAGGAACAAAGAGAGTTTG[A/G]GAATCAATGCCTGGCTA
ATAATAGG
rs558025 is set for in SEQ ID NO. 2 (polymorphic
site bracketed):
TTGTGGAAAAAGATAGATCAGGTCC[C/T]ACTTGAAGACAAAGTTGC
TCTCAAC
rs7201082 is set for in SEQ ID NO. 3 (polymorphic
site bracketed):
CAGAGGGAGAGGACAGTAACCAATA[C/T]CGCTCCTTCTACATGATC
AGTGTTC

According to some embodiments, the classification algorithm comprises a non-linear classification algorithm.

According to some embodiments, the classification algorithm may be derived from a machine learning process.

According to some embodiments, the machine learning process includes preprocessing of the acquired signals by for example normalization, filtering, noise reduction, SNR optimization, domain transformations, statistical analysis, spectral analysis, wavelet analysis, or the like.

According to some embodiments, the machine learning process includes a process of feature selection and dimensionality reduction wherein a great plurality of features, including numerous polymorphic sites and clinical features undergo feature selection and dimensionality reduction to obtain a smaller amount of features relevant to providing an efficient prediction of the treatment response. According to some embodiments, the feature selection and dimensionality reduction techniques are selected from the group consisting of Multi Dimensional Scaling (MDS), Principal Component Analysis (PCA), Least Absolute Shrinkage and Selection Operator (LASSO), Sparse PCA (SPCA), Fisher Linear Discriminant Analysis (FLDA), minimum Redundancy Maximum Relevance (mRMR), Sparse FLDA (SFLDA), Kernel PCA (KPCA), ISOMAP, Locally Linear Embedding (LLE), Laplacian Eigenmaps, Diffusion Maps, Hessian Eigenmaps, Independent Component Analysis (ICA), Factor analysis (FA), Dimensionality Reduction (HDR), Sure Independence Screening (SIS), Fisher score ranks, t-test rank, Mann-Whitney U-test and any combination thereof, or as known and accepted in the art. Each possibility is separate embodiment. According to some embodiments, the feature selection technique applied during the machine learning process is Least Absolute Shrinkage and Selection Operator (LASSO).

According to some embodiments, the machine learning may be combined with expert knowledge. It is understood that this is a prerequisite for reliable feature selection since the number of possible features, especially the number of genetic features will always exceed the number of subjects included in the machine learning process. According to some embodiments, approximately 500,000 SNPs were included in the machine learning process. According to some embodiments, 200-300 clinical features were included in the machine learning process. Advantageously, feature selection based on a combination of mathematical feature selection techniques and expert knowledge enables reliable feature selection. According to some embodiments, the expert knowledge applied includes identification of the genetic feature as belonging to a related pathway, as being positioned within or in proximity to a candidate gene, as being epigenetic markers of regulatory elements and the like.

According to some embodiments, processing the at least one clinical feature and/or the nucleotide identity of the at least one (or at least two) polymorphic includes classification into at least two or more classes, for example efficient and non-efficient. According to some embodiments, suitable classifiers include but are not limited to: Nearest Shrunken Centroids (NSC), Classification and Regression Trees(CART), ID3, C4.5, Multivariate Additive regression splines (MARS), Multiple additive regression trees(MART), Nearest Centroid (NC), Shrunken Centroid Regularized Linear Discriminate and Analysis (SCRLDA), Random Forest, Random Jungle, Boosting, Bagging Classifier, AdaBoost, RealAdaBoost, LPBoost, TotalBoost, BrownBoost, MadaBoost, XGBoost, LogitBoost, GentleBoost, RobustBoost, Support Vector Machine (SVM), kernelized SVM, Linear classifier, Quadratic Discriminant Analysis (QDA) classifier, Naive Bayes Classifier and Generalized Likelihood Ratio Test (GLRT) classifier with plug-in parametric or non-parametric class conditional density estimation, k-nearest neighbor, Radial Base Function (RBF) classifier, Multilayer Perceptron classifier, Bayesian Network (BN) classifier, multi-class classifier adapted from binary classifier with one-vs-one majority voting, one-vs-rest, Error Correcting Output Codes, hierarchical multi-class classification, Committee of classifiers or other classifiers known and accepted in the art or any combination thereof. Each possibility is separate embodiment. According to some embodiments, the non-linear classification algorithm is an ensemble of classification and regression trees. According to some embodiments, the non-linear classification algorithm is a random forest classifier or a boosting framework.

According to some embodiments, applying the classification algorithm further includes proving a graded score relating the level of treatment efficacy.

According to some embodiments, the method predicts the efficacy of the psychiatric treatment with at least 50% accuracy, at least 55% accuracy, at least 60 percent accuracy, at least 62% accuracy, at least 65% accuracy or at least 70% accuracy. Each possibility is a separate embodiment. According to some embodiments, the method predicts the efficacy of the antidepressant treatment with at least 50% accuracy, at least 55% accuracy, at least 60 percent accuracy, at least 62% accuracy, at least 65% accuracy or at least 70% accuracy. Each possibility is a separate embodiment. According to some embodiments, the method predicts the efficacy of the citalopram treatment with at least 50% accuracy, at least 55% accuracy, at least 60 percent accuracy, at least 62% accuracy, at least 65% accuracy or at least 70% accuracy. Each possibility is a separate embodiment.

According to some embodiments, the method further includes displaying or otherwise communicating the classification results. According to some embodiments, the classification results may be displayed in a plurality of formats including printout, visual display cues, acoustic cues or the like. Each possibility is separate embodiment.

According to some embodiments, the method further includes predicting the risk of side effects resulting from treating the subject with the psychiatric drug. According to some embodiments, the side effects include sexual side effects resulting from the treatment. According to some embodiments, the side effects include sexual side effects resulting from treatment with citalopram. According to some embodiments, predicting the sexual side effects of the citalopram treatment comprises obtaining the patients age and gender and applying the classification algorithm thereon. According to some embodiments, sexual side effects include but are not limited to sexual desire, orgasm, erection.

According to some embodiments, the genetic material as analyzed herein for determining the presence of SNPs within genetic loci of a subject treated with or about to be treated with antidepressant drugs, may be extracted from virtually any body sample, such as blood (other than pure red blood cells), tissue material and the like by a variety of techniques such as that described by Maniatis, et. al. (Molecular Cloning: A Laboratory Manual, Cold Spring Harbor, N.Y., pp. 280-281, 1982). Convenient tissue samples include whole blood, semen, saliva, tears, urine, fecal material, sweat, buccal material, skin and hair. For assay of cDNA or mRNA, the tissue sample must be obtained from an organ in which the target nucleic acid is expressed. According to certain embodiments, the genomic DNA sample is obtained from whole blood samples or EBV-transformed lymphoblast lines.

Typically, the sample obtained from the subject is processed before the detecting step, e.g. the DNA in the cell or tissue is separated from other components of the sample, and the target DNA is amplified as described herein below. All samples obtained from a subject, including those subjected to any sort of further processing, are considered to be obtained from the subject.

If the extracted sample is impure, it may be treated before analysis with an amount of a reagent effective to open the cell membranes of the sample, and to expose and/or separate the strand(s) of the nucleic acid(s). This lysing and nucleic acid denaturing step exposes and separates the strands.

According to some embodiments, there is provided a method for identifying a subject as being resistant to psychiatric treatment, the method comprising obtaining a sample comprising genetic material from the subject; detecting a nucleotide identity of at least one polymorphic site in the genetic material; processing the nucleotide identity of the at least one polymorphic site by applying a classification algorithm, the classification algorithm configured to provide a graduated score indicative of the subject's treatment resistance.

The term “expert knowledge” as used herein refers to knowledge acquired by continuous experience and through professional literature.

The term “biological model” as used herein refers to models based on biologically derived data.

The term “feature selection algorithm” as used herein refers to a method for identifying relevant and fewer predictors (features) with which to perform classification or regression predictions. According to some embodiments, either one of the “feature selection” and “feature extraction”, or both, may be used for feature reduction.

The term “feature meta-ranking” as used herein refers to ranking the features based on their importance, and overall effect on the prediction of the model.

The term “machine learning algorithm” as used herein refers to a construction of a method (algorithm) that can learn from and make predictions on data.

The term “ensemble predictor” as used herein refers to combining two or more prediction models, in order to improve the prediction model.

The term “exponential modeling” as used herein refers to a model that fits the data exponentially, this will suit cases where the data change by a fixed (or close to fixed) percentage.

The term “meta-analysis” as used herein refers to method for combining data from multiple studies or models.

According to some embodiments, there is provided a method for identifying a subject as being resistant to psychiatric treatment, the method comprising obtaining at least one clinical feature of the patient and/or obtaining a sample comprising genetic material from the subject; detecting a nucleotide identity of at least at least one polymorphic site in the genetic material; processing the at least one clinical feature and/or the nucleotide identity of the at least one polymorphic site by applying a classification algorithm, the classification algorithm configured to provide a graduated score indicative of the subject's treatment resistance.

According to some embodiments, there is provided a method for identifying a subject as being resistant to psychiatric treatment, the method comprising obtaining at least one clinical feature of the patient; obtaining a sample comprising genetic material from the subject; detecting a nucleotide identity of at least two polymorphic sites in the genetic material; processing the at least one clinical feature and the nucleotide identity of the at least two polymorphic sites by applying a classification algorithm, the classification algorithm configured to provide a graduated score indicative of the subject's treatment resistance.

According to some embodiments, the subject in need of the psychiatric treatment may suffer from a psychiatric disorder selected from the group consisting of depression, attention deficit disorder, schizophrenia, bipolar disorder, anxiety disorders, alcoholism, eating disorders such as anorexia and bulimia, phobias, dissociative disorders, insomnia, and borderline personality disorder or any combination thereof. According to some embodiments, the subject is suffering from depression and the psychiatric treatment is treatment with an antidepressant.

According to some embodiments, the at least one clinical feature is selected from the group consisting of: severity level of problems in the musculoskeletal and/or integument system, severity level of problems in the neurological system, employment status, residence, age, fear of having an anxiety attack, reported feeling of unease and any combination thereof. According to some embodiments, the method includes obtaining at least three of the above-mentioned clinical features. According to some embodiments, the clinical features incorporated into the method may include most or all of the above-described clinical features.

According to some embodiments, the at least one and/or the at least two polymorphic sites are selected from the group consisting of: rs1057079, rs10892629, rs12625531, rs1303860, rs1349620, rs1361038, rs1475774, rs1488467, rs16912741, rs16959216, rs17049528, rs1854696, rs1873906, rs1891932, rs3122155, rs4845882, rs530296, rs625109, rs6913639, rs7203315, rs732123, rs948025 and any combination thereof.

rs1057079 is set for in SEQ ID NO. 4 (polymorphic
site bracketed):
ACCTGCTGGATGCTGAATTAACTGC[A/G]ATGGCAGGAGAGAGTTAC
AGTCGGG
rs10892629 is set for in SEQ ID NO. 5 (polymorphic
site bracketed):
CAAGTTACTTAACCTGTTGGCCTAT[C/T]AGTTTTCCCTCTTGTAAAAT
GGAGA
rs12625531 is set for in SEQ ID NO. 6 (polymorphic
site bracketed):
CCTATCCTAGACCTACTGAATTAGA[A/G]TCCACATTTTAATAAGATT
CTGGGT
rs1303860 is set for in SEQ ID NO. 7 (polymorphic
site bracketed):
AATGGACAAATTTGTCTGTGATCCA[C/T]ATATTCTCTCTTCCTCTAGC
TTAGG
rs1349620 is set for in SEQ ID NO. 8 (polymorphic
site bracketed):
AATATCAAATGGGTTGGGTGAGATC[A/G]CTTATGCAATATAATCCCA
GCACTT
rs1361038 is set for in SEQ ID NO. 9 (polymorphic
site bracketed):
ATGGGAAATGGAATACCATAAAATT[A/G]TCATATGTTGAGCCCAAA
ATGATAG
rs1475774 is set for in SEQ ID NO. 10 (polymorphic
site bracketed):
AATACTTGTTTTCTAATGATTCAAG[A/G]TACACAAATTTTATTTAATG
CACAA
rs1488467 is set for in SEQ ID NO. 11 (polymorphic
site bracketed):
CATACAGTTGGCCCTCTATATCCCT[C/G]TATCTGTGAGTTCAGTGGA
TTCAAA
rs16912741 is set for in SEQ ID NO. 12 (polymorphic
site bracketed):
TTCTGTAGTTAATAAAGTTAACACT[A/G]TTTCATGATGGAGGCTGCC
CCAGCT
rs16959216 is set for in SEQ ID NO. 13 (polymorphic
site bracketed):
TTCTGGGTTTGGGTTAGTAGTTTCA[C/T]GAAGATACACCACCCTTCCT
CTTCC
rs17049528 is set for in SEQ ID NO. 14 (polymorphic
site bracketed):
ACATGCTCCTTCACCTTTGAGCTTC[A/G]GCCAGGGAGAAAAACACAT
ATTAGA
rs1854696 is set for in SEQ ID NO. 15 (polymorphic
site bracketed):
GAGTGTATTTGTAAAACATGTTGTT[C/T]GCCCCAGTAAATGTATTCA
TAAACC
rs1873906 is set for in SEQ ID NO. 16 (polymorphic
site bracketed):
CACAACTAAGTGCTAGGGATAACAT[A/G]GTGAAAAAATTAAAAAAC
AGAGAAG
rs1891932 is set for in SEQ ID NO. 17 (polymorphic
site bracketed):
ATCGCCTACGCCTGCAGTCAGTTAT[C/T]CTTCACTCAGACCACCAGC
CCTCTG
rs3122155 is set for in SEQ ID NO. 18 (polymorphic
site bracketed):
GAAGGGGTTAATGGTCCCCAAGCAA[A/C/G]TCTTTAACACAGCAGGG
CACATATT
rs4845882 is set for in SEQ ID NO. 19 (polymorphic
site bracketed):
ATGAATGCATATTGTAAAACCAAGC[A/G]GAGGACACAGAATGGCTC
CTCCCAC
rs530296 is set for in SEQ ID NO. 20 (polymorphic
site bracketed):
TGGAGAAACCCCGTCTCTACCAAAA[A/G]TATAAGATTAGCCAGGCC
TGGTGGG
rs625109 is set for in SEQ ID NO. 21 (polymorphic
site bracketed):
CAAGCATACTACAGCTTAATGTTTG[C/T]CCAGATGTGGGGGAAACTT
TGTTTT
rs6913639 is set for in SEQ ID NO. 22 (polymorphic
site bracketed):
AGATCAATTGAGTTAATGTATGTAA[C/T]GTACTTGGCACAGAGCTTG
GCCCAT
rs7203315 is set for in SEQ ID NO. 23 (polymorphic
site bracketed):
TAATGTCCTAAATCTGTAATGGAGT[A/G/T]AATCAACAAGACACTTG
CAAACAAA
rs732123 is set for in SEQ ID NO. 24 (polymorphic
site bracketed):
ACTCAGTTTCTTTACATAGCTATAG[A/G]ATGAGGGCGTAGTCCTAGA
GTGCTC
rs948025 is set for in SEQ ID NO. 25 (polymorphic
site bracketed):
TGTTGTGAAGATTAAATGAGATGGT[G/T]CTTAAATGTTACTTAGTAG
TAGTAG

According to some embodiments, the method includes detecting the nucleotide identity of at least four of the above polymorphic sites. According to some embodiments, the method includes detecting the nucleotide identity of most (e.g. 10) or all of the above polymorphic sites.

According to some embodiments, a subject being resistant to antidepressant treatment comprises a subject being resistant to at least two antidepressant medications selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone and vortioxetine and any combination thereof.

According to some embodiments, the classification algorithm comprises a non-linear classification algorithm.

According to some embodiments, the classification algorithm may be derived from a machine learning process.

According to some embodiments, the machine learning process includes preprocessing of the acquired signals by for example normalization, filtering, noise reduction, SNR optimization, domain transformations, statistical analysis, spectral analysis, wavelet analysis, or the like.

According to some embodiments, the machine learning process includes a process of feature selection and dimensionality reduction wherein a great plurality of features, including numerous polymorphic sites and/or clinical features undergo feature selection and dimensionality reduction to obtain a smaller amount of features relevant to providing an efficient prediction of the treatment response. According to some embodiments, the feature selection and dimensionality reduction techniques are selected from the group consisting of Multi Dimensional Scaling (MDS), Principal Component Analysis (PCA), Least Absolute Shrinkage and Selection Operator (LASSO), minimum Redundancy Maximum Relevance (mRMR), Sparse PCA (SPCA), Fisher Linear Discriminant Analysis (FLDA), Sparse FLDA (SFLDA), Kernel PCA (KPCA), ISOMAP, Locally Linear Embedding (LLE), Laplacian Eigenmaps, Diffusion Maps, Hessian Eigenmaps, Independent Component Analysis (ICA), Factor analysis (FA), Dimensionality Reduction (HDR), Sure Independence Screening (SIS), Fisher score ranks, t-test rank, Mann-Whitney U-test and any combination thereof, or as known and accepted in the art. Each possibility is separate embodiment. According to some embodiments, the feature selection technique applied during the machine learning process is Least Absolute Shrinkage and Selection Operator (LASSO).

According to some embodiments, the machine learning may be combined with expert knowledge. It is understood that this is a prerequisite for reliable feature selection since the number of possible features, especially the number of genetic features will always exceed the number of subjects included in the machine learning process. According to some embodiments, approximately 500,000 SNPs were included in the machine learning process. According to some embodiments, 200-300 clinical features were included in the machine learning process. Advantageously, feature selection based on a combination of mathematical feature selection techniques and expert knowledge enables reliable feature selection. According to some embodiments, the expert knowledge applied includes identification of the genetic feature as belonging to a related pathway, as being positioned within or in proximity to a candidate gene, as being indicative of an epigenetic marker of regulatory elements and the like.

According to some embodiments, processing the at least one clinical feature and/or the nucleotide identity of the at least one polymorphic site includes classification into at least two or more classes, for example efficient and non-efficient. According to some embodiments, suitable classifiers include but are not limited to: Nearest Shrunken Centroids (NSC), Classification and Regression Trees(CART), ID3, C4.5, Multivariate Additive regression splines (MARS), Multiple additive regression trees(MART), Nearest Centroid (NC), Shrunken Centroid Regularized Linear Discriminate and Analysis (SCRLDA), Random Forest, Random Jungle, Boosting, Bagging Classifier, AdaBoost, RealAdaBoost, LPBoost, TotalBoost, BrownBoost, MadaBoost, XGBoost, LogitBoost, GentleBoost, RobustBoost, Support Vector Machine (SVM), kernelized SVM, Linear classifier, Quadratic Discriminant Analysis (QDA) classifier, Naive Bayes Classifier and Generalized Likelihood Ratio Test (GLRT) classifier with plug-in parametric or non-parametric class conditional density estimation, k-nearest neighbor, Radial Base Function (RBF) classifier, Multilayer Perceptron classifier, Bayesian Network (BN) classifier, multi-class classifier adapted from binary classifier with one-vs-one majority voting, one-vs-rest, Error Correcting Output Codes, hierarchical multi-class classification, Committee of classifiers or other classifiers known and accepted in the art or any combination thereof. Each possibility is separate embodiment. According to some embodiments, the non-linear classification algorithm is an ensemble of classification and regression trees. According to some embodiments, the non-linear classification algorithm is a random forest classifier or a boosting framework.

According to some embodiments, applying the classification algorithm further includes proving a graded score relating the level of treatment efficacy.

According to some embodiments, the method predicts the efficacy of the psychiatric treatment with at least 50% accuracy, at least 55% accuracy, at least 60 percent accuracy, at least 62% accuracy, at least 65% accuracy or at least 70% accuracy. Each possibility is a separate embodiment. According to some embodiments, the method predicts the efficacy of the antidepressant treatment with at least 50% accuracy, at least 55% accuracy, at least 60 percent accuracy, at least 62% accuracy, at least 65% accuracy or at least 70% accuracy. Each possibility is a separate embodiment. According to some embodiments, the method predicts the efficacy of the citalopram treatment with at least 50% accuracy, at least 55% accuracy, at least 60 percent accuracy, at least 62% accuracy, at least 65% accuracy or at least 70% accuracy. Each possibility is a separate embodiment.

According to some embodiments, the method further includes displaying or otherwise communicating the classification results. According to some embodiments, the classification results may be displayed in a plurality of formats including printout, visual display cues, acoustic cues or the like. Each possibility is separate embodiment.

According to some embodiments, method 100 begins with selection of genomic (step 102), and clinical (step 104) specific features by using expert knowledge, biological models and feature selection algorithms. Then, the features are ranked by feature meta-ranking (step 112), and applying machine learning algorithm (step 114). Using the tuned parameters, (same or different) machine learning techniques/algorithms are selected (step 106) and parameters are selected for further feature selection (step 108) and the selected selection algorithm of step 106 is applied (step 110). Afterwards, an ensemble predictor is generated from the selection algorithm of step 110 and the machine learning algorithm of step 114 thereby obtaining an ensemble predictor enabling predicting results for replication sets (step 116) and new data sets (step 118).

The present invention further provides methods and kits for predicting a subject's response to treatment with venlafaxine, e.g. in case the subject is suffering from depression. The methods and kits are based on the identification of the SNP rs2283351 in the coiled-coil domain containing protein 63 (also referred to herein as “CCDC63”) and optionally also rs10497340 in the gene locus LOC102724081.

Venlafaxine (brand names: EFFEXOR, EFFEXOR XR, LANVEXIN, VIEPAX and TREVILOR) is an antidepressant of the serotonin-norepinephrine reuptake inhibitor (SNRI) class. This means it increases the concentrations of the neurotransmitters serotonin and norepinephrine in the synaptic cleft or synaptic gap.

The method of the invention comprises obtaining a sample comprising genetic material from the subject and detecting a nucleotide identity of rs2283351 in the genetic material. Additionally or alternatively, the method comprises detecting a gene variation present in the gene locus of CCDC63 or its regulatory elements.

Additionally or alternatively, the method includes identifying a nucleotide identity of rs10497340 in the genetic material. Additionally or alternatively, the method comprises detecting a gene variation present in the gene locus of LOC102724081 or its regulatory elements.

According to some embodiments, identification of an adenine (A) and guanine (G) (heterozygocity) in the polymorphic site of rs2283351 is indicative of lack of response to venlafaxine treatment. According to some embodiments, identification of an adenine (A) homozygocity in the polymorphic site of rs2283351 is indicative of the subject being responsive to venlafaxine treatment. According to some embodiments, identification of guanine (G) homozygocity in the polymorphic site of rs2283351 is indicative of the subject being responsive to venlafaxine treatment.

According to some embodiments, identification of an adenine (A) and guanine (G) (heterozygocity) in the polymorphic site of rs10497340 is indicative of lack of response to venlafaxine treatment. According to some embodiments, identification of guanine (G) homozygocity in the polymorphic site of rs10497340 is indicative of the subject being responsive to venlafaxine treatment.

According to some embodiments, an adenine (A) and guanine (G) (heterozygocity) in the polymorphic site of rs2283351 and an adenine (A) and guanine (G) (heterozygocity) in the polymorphic site of rs10497340 is indicative of lack of response to venlafaxine treatment.

According to some embodiments, a guanine (G) (homozygocity) in the polymorphic site of rs2283351 and a guanine (G) (homozygocity) in the polymorphic site of rs10497340 is indicative of response to venlafaxine treatment.

According to some embodiments, a guanine (G) (homozygocity) in the polymorphic site of rs2283351 and an adenine (A) and guanine (G) (heterozygocity) in the polymorphic site of rs10497340 is indicative of lack of response to venlafaxine treatment.

According to some embodiments, an adenine (A) and a guanine (G) (heterozygocity) in the polymorphic site of rs2283351 and guanine (G) (homozygocity) in the polymorphic site of rs10497340 is indicative of lack of response to venlafaxine treatment.

According to some embodiments, an adenine (A) (homozygocity) in the polymorphic site of rs2283351 and a guanine (G) (homozygocity) in the polymorphic site of rs10497340 is indicative of response to venlafaxine treatment.

According to some embodiments, an adenine (A) (homozygocity) in the polymorphic site of rs2283351 and an adenine (A) and guanine (G) (heterozygocity) in the polymorphic site of rs10497340 is indicative of response to venlafaxine treatment.

According to some embodiments, the method further includes processing the identified nucleotide identity of rs2283351 and rs10497340 by applying a classification algorithm, the classification algorithm is configured to provide a graduated score indicative of the treatment response to venlafaxine.

A person skilled in the art of psychiatry will find the present invention useful for deciding whether venlafaxine should be prescribed to a subject in need of antidepressant treatment.

According to some embodiments, the importance of the nucleotide identity of the rs2283351 and rs10497340 loci for predicting venlafaxine treatment response may be obtained through a machine learning process. According to some embodiments, the machine learning process includes a process of selecting the relevant genetic loci (feature selection) from a great plurality of genetic loci. According to some embodiments, the feature selection technique is selected from the group consisting of Multi Dimensional Scaling (MDS), Principal Component Analysis (PCA), Least Absolute Shrinkage and Selection Operator (LASSO), Sparse PCA (SPCA), Fisher Linear Discriminant Analysis (FLDA), minimum Redundancy Maximum Relevance (mRMR), Sparse FLDA (SFLDA), Kernel PCA (KPCA), ISOMAP, Locally Linear Embedding (LLE), Laplacian Eigenmaps, Diffusion Maps, Hessian Eigenmaps, Independent Component Analysis (ICA), Factor analysis (FA), Dimensionality Reduction (HDR), Sure Independence Screening (SIS), Fisher score ranks, t-test rank, Mann-Whitney U-test and any combination thereof, or as known and accepted in the field. Each possibility is a separate embodiment. According to some embodiments, the feature selection technique applied during the machine learning process is Least Absolute Shrinkage and Selection Operator (LASSO).

According to some embodiments, the method predicts the efficiency of venlafaxine treatment with at least 65% accuracy or at least 70% accuracy. Each possibility is a separate embodiment.

The methods and kits of the invention are directed at predicting venlafaxine treatment response, in patients treated or intended to be treated with venlafaxine, by identifying the nucleotide identity of rs2283351 in the CCDC63 gene and/or rs10497340 in the gene locus LOC102724081 from a biological specimen taken from the patients.

rs2283351 is set for in SEQ ID NO. 26 (polymorphic
site bracketed):
CATGGGTCTTTAGAAGCATTAGGAA[A/G]AGGCAACACAGCGTGGGG
TCCTCAG
rs10497340 is set for in SEQ ID NO. 27 (polymorphic
site bracketed):
CTGCAAGGGGCATCCGTGGCGAACC[A/G]AAAGATCTCTCAGTTGAA
GACCAGG

The DNA obtained from a subject, for determining the presence of polymorphisms in the genes examined is typically amplified. The primers used to amplify DNA strands including the rs2283351 and/or rs10497340 loci are oligonucleotides of sufficient length and appropriate sequence to provide initiation of polymerization. The oligonucleotide primer typically contains 12-20 or more nucleotides, although it may contain fewer nucleotides. The method of amplification is preferably PCR, or a construction of library for next generation sequencing as described herein and as is commonly used by those of ordinary skill in the art. Alternative methods of amplification can also be employed as long as the genetic locus amplified by PCR using primers of the invention is similarly amplified by the alternative means.

The amplified DNA may further be sequenced in order to evaluate the nucleotide identity of the SNP. A number of methods well known in the art can be used to carry out the sequencing reactions. One skilled in the art recognizes that sequencing is now often performed with the aid of automated methods.

Determining the presence and identity of SNPs or haplotypes which correlate with venlafaxine responsiveness may be carried out by any one of the various tools for the detection of polymorphism on a target DNA known in the art, including, but not limited to, allele-specific probes, allele specific primers, direct sequencing, next generation sequencing, DNA arrays denaturing gradient gel electrophoresis and single-strand conformation polymorphism. Preferred techniques for SNP genotyping should allow for large scale automated analysis, which does not require extensive optimization for each analyzed SNP.

The phrase “identifying a polymorphism” or “identifying a polymorphic variant” as used herein generally refers to determining which of two or more polymorphic variants exists at a polymorphic site. In general, for a given polymorphism, any individual will exhibit either one or two possible variants at the polymorphic site (one on each chromosome).

According to some embodiments, either one of the “feature selection” or “feature extraction” or both may be used for feature number reduction. It should be understood that the prediction model does not necessarily have to use the machine learning algorithms. In case of one or even two predicting SNPs are needed and available, a stand-alone model can be designed.

According to some embodiments, the selection algorithm(s) may include one or more of the following techniques and algorithms: Feature similarity, Simulated Annealing, Ants Colony, HillClimbing, Genetic Algorithm, iterated local search, PSO, Binary PSO or others.

Advantageously, the feature reduction, may facilitate shorter training times for the machine learning algorithms, simplification of the models, and provide modification ability by uses. According to some embodiments, the machine learning techniques/algorithms may include one or more of the following algoritms: Linear-Regression, KNN, K-Means, Random-Forest, SVM, Logistic-Regression, Decision-Tree, dimensionality reduction, Gradient boost and Adaboost, Nayve-Bayes.

The foregoing description of the specific embodiments will so fully reveal the general nature of the invention that others can, by applying current knowledge, readily modify and/or adapt it for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not limitation. The means, materials and steps for carrying out various disclosed functions may take a variety of alternative forms without departing from the invention.

### EXAMPLES

### Example 1

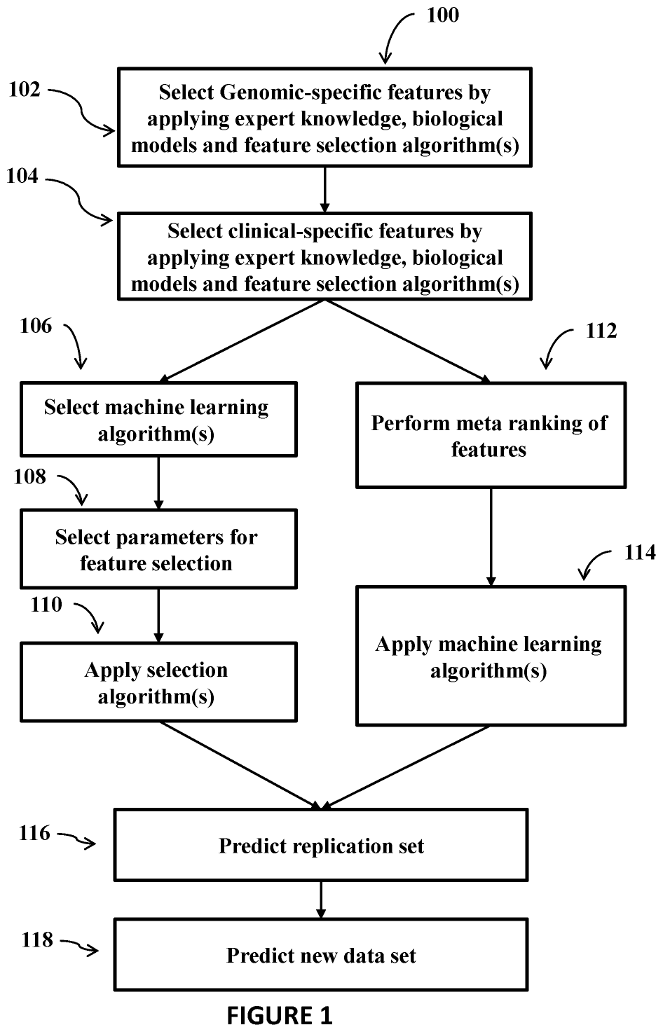

Reference is now made to FIG. 1 which illustrates a flow-chart of a prediction method 100, according to some embodiments. According to some embodiments, to handle the vast numbers of features, a multi-level feature selection is performed, both on genomic features and clinical features. According to some embodiments, the feature selection decreases the dimensionality of the computational problem, thereby reducing the number of random variables under consideration of the computational technique by obtaining a set of principal variables.

According to some embodiments, either one of “feature selection” and “feature extraction” or both may be used for feature number reduction.

According to some embodiments, method 100 begins with selection of genomic (step 102), and clinical (step 104) specific features by using expert knowledge, biological models and feature selection algorithms. Then, the features are ranked by feature meta-ranking (step 112), and applying machine learning algorithm (step 114). Using the tuned parameters, (same or different) machine learning techniques/algorithms are selected (step 106) and parameters are selected for further feature selection (step 108) and the selected selection algorithm of step 106 is applied (step 110). Afterwards, an ensemble predictor is generated from the selection algorithm of step 110 and the machine learning algorithm of step 114 thereby obtaining an ensemble predictor enabling predicting results for new test sets (step 116) and replication sets (step 118).

The above method was applied on clinical and genetic data obtained from STAR*D(NIH), including data obtained from 4,041 patients with depression from 41 psychiatric and primary care sites in the US, including follow-up data obtained 7 years after initiation of the project. The clinical data set includes 10500 clinical features including 280 enrollment features (e.g. ethnicity, medical history, personal status etc.), 9800 experimental features (e.g. dose, side effects, depression score etc.), 420 follow-up features (e.g. personal status, mood, general health etc.). The genetic data including nucleotide identity of ˜500,000 SNPs.

The data was divided into a training set for generating the ensemble predictor and two validation sets for validation of the predictor.

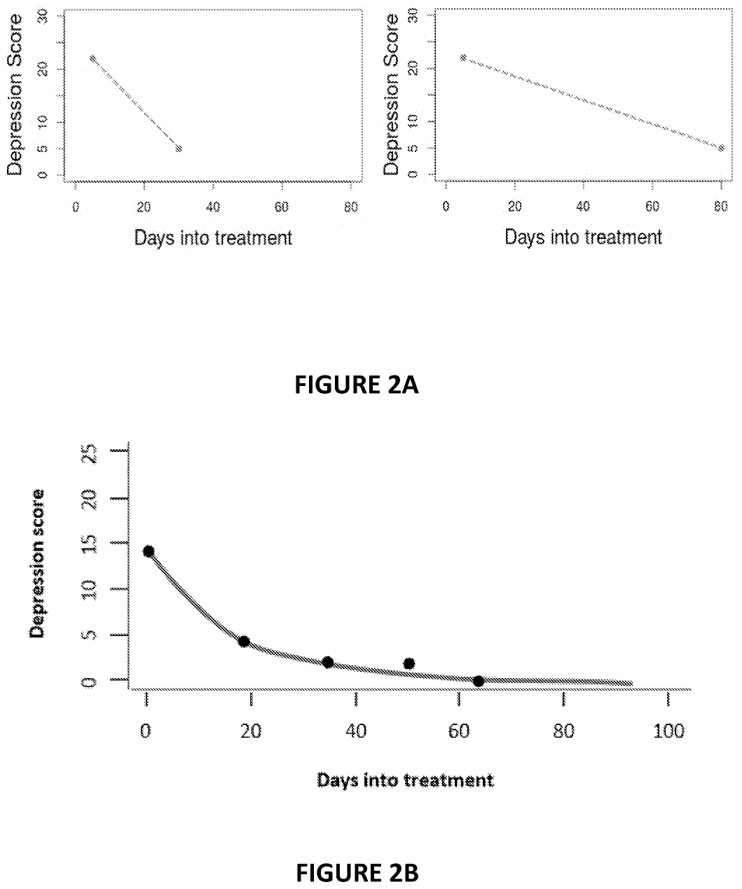

The efficacy was measured based on two data modeling methods. The first, a simple modeling of a patient's depression score based upon the definition that treatment efficacy is an improvement of 50% or more in the subject's depression score as a result of the treatment based on comparison of the first depression score (before initiation of treatment) and the last depression score (after last treatment) as illustrated in FIG. 2A. The second, based on a depression curve taking into consideration both the depression score as well as duration of treatment, the curve depicting an exponential function f(x)=e^{ax+b}, wherein a is the change in depression score (the slope), b is a normalization factor and x the duration (time) of treatment, as illustrated in FIG. 2B.

### Example 2

### Citalopram Treatment Response

The method identified 9 clinical features severity and 3 genetic features as predictors of citalopram treatment response.

The identified clinical features include: severity level of problems in the upper gastro intestine, pains or aches at different body parts, reported fear of having anxiety attack, history of psychotropic medications, poor treatment response to other antidepressants, reported troubling thoughts, employment status, residence, private health care insurance.

The identified genetic features include: rs17291388, rs558025, rs7201082.

Applying the ensemble predictor on the identified clinical and genetic features for predicting the efficacy of citalopram treatment had an accuracy of 0.62295 with a p-value of 2.95E-06 and an AUC of 0.6589.

### Example 3

### Venlafaxine Treatment Response

The method identified two genetic features as predictors of venlafaxine treatment response.

The identified genetic features include: rs2283351 and rs10497340.

The predictive values of SNPs were measured for rs2283351 and rs10497340 SNPs.

The predictive values of the identified genetic feature SNP rs2283351 for predicting the efficacy of venlafaxine treatment had an accuracy of 72.5% with a p-value of 0.0079, AUC of 0.7306, sensitivity of 61.9%, specificity of 84.21%, positive predictive value of 81.52% and negative predictive value of 66.7.

The predictive values of the identified genetic features SNP rs10497340 for predicting the efficacy of venlafaxine treatment had an accuracy of 55% with a p-value of 0.438, AUC of 0.4311, sensitivity of 19.05%, specificity of 94.73%, positive predictive value of 80% and negative predictive value of 51.42%.

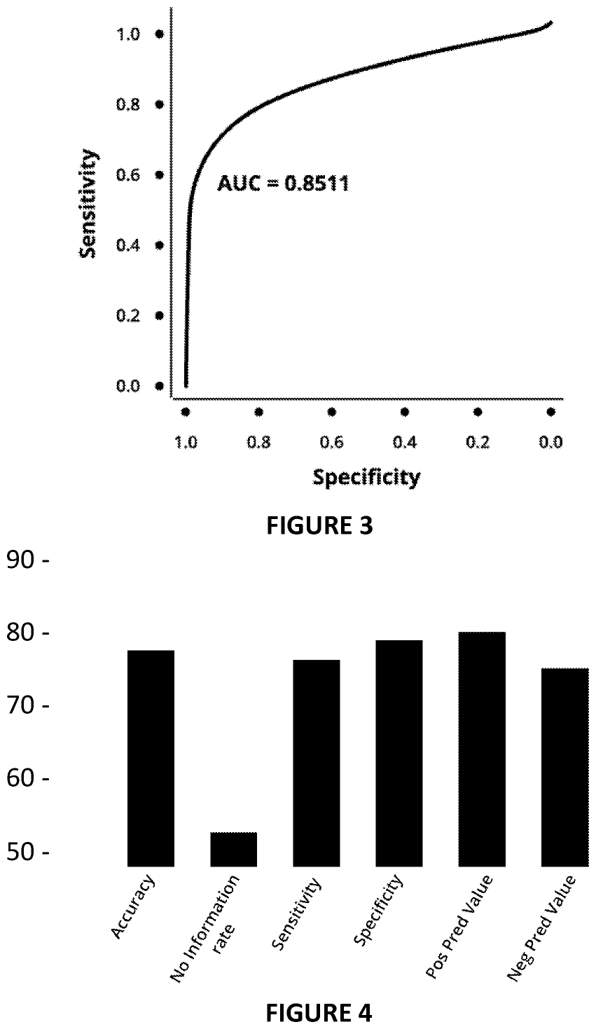

It was clearly shown that predictive value of SNPs rs2283351 is stronger than that of rs10497340. The identified predictive value of the genetic features including both rs2283351 and rs10497340 SNPs for predicting the efficacy of venlafaxine treatment had an accuracy of 0.775 with a p-value of 0.001 and an AUC of 0.8511 as seen in FIG. 3. The accuracy (77.5%), sensitivity (76.19%), specificity (78.95), positive predictive value (80%) and negative predictive value (75%) of the predictor are summarized in FIG. 4.

### Example 4

### Treatment Resistance

The method identified 7 clinical severity features and 22 genetic features as predictors of treatment resistance.

The identified clinical features include: level of problems in the musculoskeletal/integument system, severity level of problems in the neurological system, employment status, resistance to previous treatment, age, feared of having an anxiety attack, reported feeling of unease.

The identified genetic features include: rs1057079, rs10892629, rs12625531, rs1303860, rs1349620, rs1361038, rs1475774, rs1488467, rs16912741, rs16959216, rs17049528, rs1854696, rs1873906, rs1891932, rs3122155, rs4845882, rs530296, rs625109, rs6913639, rs7203315, rs732123, rs948025.

Applying the ensemble predictor on the identified clinical and genetic features for predicting treatment resistance had an accuracy of 0.5998 with a p-value of 7.24E-06 and an AUC of 0.6381.

1. A method for predicting citalopram treatment response for a subject in need thereof, the method comprising:
obtaining at least one clinical feature of the subject;
obtaining a sample comprising genetic material from the subject;
detecting a nucleotide identity of at least two polymorphic sites in the genetic material, wherein the at least two polymorphic sites are selected from the group consisting of: rs17291388, rs558025, and rs7201082; and
processing the at least one clinical feature and the nucleotide identity of the at least two polymorphic sites by applying a classification algorithm, the classification algorithm configured to provide a graduated score indicative of the treatment response to citalopram.

2. The method of claim 1, wherein the at least one clinical feature is selected from the group consisting of severity level of problems in the upper gastro intestine, reported pains or aches in different body parts, reported fear of having an anxiety attack, having a history of psychotropic medications, having a poor treatment response to other antidepressants, reported troubling thoughts, and any combination thereof.

3. The method of claim 2, wherein the at least one clinical feature is a plurality of clinical features, comprising severity level of problems in the upper gastro intestine, reported pains or aches in different body parts, reported fear of having an anxiety attack, having a history of psychotropic medications, having a poor treatment response to other antidepressants and reported troubling thoughts.

4. The method of claim 1, further comprising obtaining personal and/or demographic information regarding the subject, wherein the personal information obtained relates to employment status, having private healthcare insurance, age, marital status, residence, and any combination thereof.

5. The method of claim 1, further comprising determining sexual side effects of the citalopram treatment, wherein determining the sexual side effects of the citalopram treatment comprises obtaining the patients' age and gender and applying the classification algorithm thereon.

6. The method of claim 1, wherein the classification algorithm comprises an ensemble of classification and regression trees, wherein said ensemble of classification and regression trees, comprises a random forest classifier or a boosting framework.

7. A method for identifying a subject as being resistant to antidepressant treatment, the method comprising:
obtaining at least one clinical feature of the subject;
obtaining a sample comprising genetic material from the subject;
detecting a nucleotide identity of at least two polymorphic sites in the genetic material; and
processing the at least one clinical feature and the nucleotide identity of the at least two polymorphic sites by applying a classification algorithm, the classification algorithm configured to provide a graduated score indicative of the subject's treatment resistance.

8. The method of claim 7, wherein the at least one clinical feature is selected from the group consisting of: severity level of problems in the musculoskeletal/integument system, severity level of problems in the neurological system, avoiding doing something because of fear from having an anxiety attack, fear of having an anxiety attack when traveling in a bus, train, or plane, being jumpy and easily startled because of having experienced a traumatic event, and any combination thereof.

9. The method of claim 7, further comprising obtaining personal and/or demographic information regarding the subject, wherein the personal information obtained relates to employment status, residence, age, having private healthcare insurance, marital status, and any combination thereof.

10. The method of claim 7, wherein the at least two polymorphic sites are selected from the group consisting of: rs1057079, rs10892629, rs12625531, rs1303860, rs1349620, rs1361038, rs1475774, rs1488467, rs16912741, rs16959216, rs17049528, rs1854696, rs1873906, rs1891932, rs3122155, rs4845882, rs530296, rs625109, rs6913639, rs7203315, rs732123, and rs948025.

11. The method of claim 7, wherein resistance to antidepressant treatment comprises resistance to at least two of the antidepressant medications selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone, and vortioxetine.

12. The method of claim 7, wherein the classification algorithm comprises an ensemble of classification and regression trees, wherein said ensemble of classification and regression trees, comprises a random forest classifier or a boosting framework.

13. A method for predicting venlafaxine treatment response for a subject in need thereof, the method comprising:
obtaining a sample comprising genetic material from the subject; and
identifying a nucleotide identity of polymorphic site rs2283351 in the genetic material.

14. The method of claim 13, further comprising identifying a nucleotide identity of polymorphic site rs10497340 in the genetic material.

15. The method of claim 14, further comprising processing the identified nucleotide identity of rs2283351 and rs10497340 by applying a classification algorithm, the classification algorithm is configured to provide a graduated score indicative of the treatment response to venlafaxine.

16. The method of claim 15, further comprising prescribing and/or administering venlafaxine to the subject if the nucleotide identity of polymorphic site rs2283351 is adenine/adenine or guanine/guanine.

17. The method of claim 13, further comprising prescribing and/or administering venlafaxine to the subject (i) if the nucleotide identity of polymorphic site rs2283351 is guanine/guanine and the nucleotide identity of polymorphic site rs10497340 is guanine/guanine; (ii) if the nucleotide identity of polymorphic site rs2283351 is adenine/adenine and the nucleotide identity of polymorphic site rs10497340 is guanine/guanine; or (iii) if the nucleotide identity of polymorphic site rs2283351 is adenine/adenine and the nucleotide identity of polymorphic site rs10497340 is adenine/guanine.

18. The method of claim 13, further comprising refraining from prescribing and/or administering venlafaxine to the subject if the nucleotide identity of polymorphic site rs2283351 is guanine/adenine.

19. The method of claim 13, further comprising refraining from prescribing and/or administering venlafaxine to the subject (i) if the nucleotide identity of polymorphic site rs2283351 is guanine/guanine and the nucleotide identity of polymorphic site rs10497340 is adenine/guanine; (ii) if the nucleotide identity of polymorphic site rs2283351 is adenine/guanine and the nucleotide identity of polymorphic site rs10497340 is guanine/guanine; or (iii) if the nucleotide identity of polymorphic site rs2283351 is adenine/guanine and the nucleotide identity of polymorphic site rs10497340 is adenine/guanine.
